(19) United States
(12) Patent Application Publication
(10) Pub. No.: US 2010/0068721 A1
(43) Pub. Date: Mar. 18, 2010

(54) Method and system for protection and cross protection of algae and cyanobacteria from virus an bacteriophage infections

(75) Inventors: Jonathan Gressel, Rehovot (IL); Ofra Chen, Rehovot (IL); Michael Danon, Bnei-Atarot (IL)

Correspondence Address: John Dodds, 1707 N St. NW, Washington, DC, 20036, US

(73) Assignee: TransAlgae Ltd, Rehovot (IL)

(21) Appl. No.: 12/584,572

(22) Filed: Sep. 8, 2009

Related U.S. Application Data
(60) Provisional application No. 61/191,452, filed on Sep. 9, 2008.

(51) Int. Cl. C12Q 1/68; C12N 15/74; C12N 1/13

(52) U.S. Cl. 435/6; 435/471; 435/257.2

(57) ABSTRACT

Virus contaminations of bioreactors can cause considerable losses in the industry and prevention of such contaminations is usually a major concern, especially in continuous cultures and particularly in outdoor/uncovered operations such as ponds or “racing ponds”. Use of transgenic algae/cyanobacteria harboring introgressed virus/phage DNA fragments, cultured in these bioreactors/ponds will provide protection to a range of viruses/phages. Molecular mechanisms such as lysogeny and post transcriptional gene silencing (PTGS) are being exploited to produce protected algae/cyanobacteria with cross protective resistance against various viruses/phage, thus gaining bioreactor stability.

### PRIORITY

This application claims priority of the U.S. provisional application No. 61/191,452 filed on Sep. 9, 2008.

### FIELD OF THE INVENTION

This invention relates in general to immunizing algae and cyanobacteria grown in cultures, photo-bioreactors and/or in ponds against viral infections, aiming to ensure reactor operation with a failsafe mechanism by establishment of immune population. More specifically, this invention relates to the method and system that utilize transgenic algae and cyanobacteria, which are immune/resistant to virus/phage infections, for the purpose of improved bioreactor stability and performance.

### BACKGROUND OF THE INVENTION

A major concern in algae/cyanobacteria culture facilities is their susceptibility to virus/phage infections, which despite the use of various preventive techniques, occasionally requires reactor shutdown and expensive/hazardous cleaning and disinfection steps. This invention described below provides an inexpensive and safe method to protect algae and cyanobacteria in bioreactors or ponds from virus/phage infection.

In previous studies, the coat protein genes of many plant viruses have been transformed into a wide range of plant species to obtain viral protection. In some cases the expression of the protein has been responsible for the resistance, but in other of cases the resistance has been demonstrated to occur at the RNA level (Lindbo and Dougherty, 1992; Baulcombe, 1996). The expression of virus-derived sense or antisense RNA in transgenic plants conferring RNA-mediated virus resistance appears to induce a form of post transcriptional gene silencing (PTGS) (Baulcombe, 1996; Stam et al., 1997). The PTGS mechanism is typified by the highly specific degradation of both the transgene mRNA and the target RNA, which contains either the same or complementary nucleotide sequences. If the transgene contains viral sequences, then virus genomic RNA containing these sequences cannot accumulate in the plant (Lindbo and Dougherty, 1992; Baulcombe, 1996). However, there has been little if any work done on viral immunization of algae. Viral infection of bacteria and cyanobacteria is known to be either lytic, causing destruction of the host cell, or lysogenic, in which the viral genome is instead stably maintained as a prophage within its host. Cyanobacteria can be resistant to lytic infection by co-occurring cyanophages (Wommack and Colwell, 2000). Lysogeny was shown to occur in natural populations of the cyanobacterium Synechococcus. That lysogeny confers immunity to infection by related viruses (Ackermann, 1987). It has been demonstrated that newly isolated Synechococcus clones are generally resistant to cyanophages found within the same environment and this may account for the resistance to viral infection seen in common forms of autotrophic picoplankton (Waterbury and Valois, 1993; McDaniel et al., 2002).

### SUMMARY OF THE INVENTION

This invention relates to the idea that cultured alga/cyanobacteria engineered with genomic fragments of algae- or cyanobacteria-attacking viruses, under the control of various promoters, renders protection to this and cross-protection to other viruses. The activation of the resistance phenomenon by engineered algae/cyanobacteria, prevents the expression of a subsequent challenge by a pathogenic virus, by which it will prevent some virus diseases.

The method of this invention provides a solution to above described problems of the current technology. The method of this invention is based on genetic manipulations using random viral genomic fragments expressed in algae and cyanobacteria conferring protection to this and cross protection to other viruses, by activation of lysogeny and/or post transcriptional gene silencing (PTGS) mechanisms in the host algae/cyanobacteria cells.

The present invention relates in general to transcription of viral/phage genes that render algae and cyanobacteria resistant to viruses. Random fragments of algal or cyanobacterial viral or phage genomes are inserted into algae and cyanobacteria cells, resistant colonies are selected using virus/phage resistance screen.

In one embodiment the target algal/cyanobacterial strain is transformed with a restriction enzyme produced mix of algal or cyanobacterial viral/phage nucleic acid fragments of the pathogen and the culture is screened and selected for different resistant strains where resistance is conferred by different fragments. Those strains that are as nearly as fit as the wild type are kept separately but are also mixed in the feeder reactors used to seed bioreactors and ponds, such that mixed modes of resistance to the same pathogen are cultivated. This is a major step to delay the evolution of resistant viruses/phages, as those evolving resistance to one mechanism will be controlled by the others.

It is not simple to confer resistance to a large number of viral/phage pathogens to a single strain of algae cultured in a production facility. Thus, in an another embodiment various combinations of transgenic strains of the same algal/cyanobacterial species bearing transgenes conferring resistance to different pathogens are grown together in the ponds or photo-bioreactors. In this manner, if the facility is infected with one pathogen, the culture will quickly be taken over by the resistant strain, with a minimum lag period.

This method enables response to unknown viruses/phages species infecting algae or cyanobacteria emerging in the bioreactor, since the resistance is conferred by random fragments of the algae or cyanobacteria virus/phage inserted into the algae and cyanobacteria. These algae and cyanobacteria infecting viruses/phages are composed from either DNA or RNA single or double stranded genomes. According to this invention there is no need in prior knowledge of the virus/phage genome in order to transgenically confer algae and cyanobacteria virus resistance. Furthermore, this invention provides protection to the specific virus, and cross protection to other algae and cyanobacteria viruses/cyanophages when its genome fragments are expressed in the algae/cyanobacteria. In addition, this invention enables the identification of new genes that harbor virus resistance.

### BRIEF DESCRIPTION OF THE FIGURES

FIG. 1. Schematic illustration of the PBCV-1 virus DNA fragments cloned under the RbcS-Hsp70 promoters in the pSI103 expression vector

FIG. 2A. Schematic illustration of the Syn9 virus DNA fragments cloned under the RbcS promoter in the pCB4 expression vector

FIG. 2B. Schematic illustration of the p60 virus DNA fragments cloned under the RbcS promoter in the pCB4 expression vector

### DETAILED DESCRIPTION OF THE INVENTION

Growing microalgae and cyanobacteria for production of oil and other products requires sustaining continuous high cell density cultures. These cultures are susceptible to viral attack and can collapse. Viral infection is thought to depend on host population density, and indeed such infections occur in natural conditions in cases of algal blooms, where hosts are abundant. Viruses are considered important as an evolutionary control of blooms, and dense cultures are analogous to blooms. Common natural phytoplankton populations that are known to be susceptible to viral infection include the chrysophyte Aureococcus anophagefferans (Milligan and Cosper, 1994), Phaeocystis pouchetii (Jacobsen et al, 1996) and Emiliana huxleyi (Bratbak, 1993). Marine cyanobacteria are also subjected to bacteriophage infection, and both hosts and phages were shown to have co-evolved to selection pressures imposed upon one another (Bailey et al., 2004). Sequences from cultured cyanophages fall within a few well-defined clusters (Zhong et al., 2002; Marston and Sallee, 2003), and all of these clusters are within a well-supported monophyletic group of cultured Synechococcus phage (Short and Suttle, 2005). About 40% of cultured marine bacteria are lysogens (bacteria that harbor prophage and can be induced to produce lytic viruses) (Jiang and Paul, 1998). In seawater and lake water samples (Jiang, 1996; Tapper, 1998), lysogens were common, with variable abundances ranging from undetectable to almost 40% of the total bacteria, and this variability can be seasonal (Cochran and Paul, 1998).

Bearing in mind the prevalence and abundance of viruses in natural environment such as sea waters, and the specific groups of viruses matching groups of algae and cyanobacteria, it is clear that successful operation of photo-bioreactors and ponds growing algae and cyanobacteria is dependent on growing strains with lasting property of immunity or resistance to viruses and/or using efficient response to their occurrence.

Wild type natural populations of algae and cyanobacteria may serve as vectors for viruses and therefore their establishment in reactors is a considerable threat to the culture stability. One strategy to reduce infection risk carried by wild type infected algae and cyanobacteria contamination is to operate photo-bioreactors and open ponds using selective culture media with herbicides and culturing algae and cyanobacteria which are genetically modified to confer herbicide resistance. The method and the algal/cyanobacteria cells possessing such modified characters are disclosed and claimed in another patent application of our research group. This invention enables more advanced protection and cross protection of the cultured algae/cyanobacteria against newly emerging viruses in the bioreactor and open ponds.

The method consists on cloning of fragmented algal or cyanobacterial viral genomic DNA (or reverse transcribed DNA from RNA viruses) inserted into appropriate expression vectors, followed by transformation into the desired algae or cyanobacteria, and selection of the resistant transformants using the overlay method. A variety of new resistant strains with acceptable growth rate are co-cultured in feeder bioreactor photo-bioreactors and ponds. Ponds are monitored to validate that all the desirable strains remain continuously active populations despite dilution rates. Sustaining different virus resistant strains growing together prevents the virus/phage from evolving resistance to the protection method, and cross-protects them against related virus species.

The described method is advantageous for fast, inexpensive and persistent protection from viruses. Because several different strains are co-cultured in bioreactor/ponds, the virus/phage is not likely to evolve resistance to the protection method, and may give more protection against related virus species. We thus demonstrate the ability of this method to protect microalgae and cyanobacteria of viral infection.

In the various embodiments, algae and cyanobacteria were chosen from the following organisms: This is done for the following algae: Chlamydomonas reinhardtii, Pavlova lutheri, Isochrysis CS-177, Nannochloropsis oculata CS-179, Nannochloropsis like CS-246, Nannochloropsis salina CS-190, Tetraselmis suecica, Tetraselmis chuii and Nannochloris sp as representatives of all algae species. The algae come from a large taxonomical cross section of species (Table 1)

TABLE 1
Phylogeny of some of algae used

| Genus | Family | Order | Phylum | Sub-Kingdom |
|---|---|---|---|---|
| Chlamydomonas | Chlamydomonadaceae | Volvocales | Chlorophyta | Viridaeplantae |
| Nannochloris | Coccomyxaceae | Chlorococcales | Chlorophyta | Viridaeplantae |
| Tetraselmis | Chlorodendraceae | Chlorodendrales | Chlorophyta | Viridaeplantae |
| Phaeodactylum | Phaeodactylaceae | Naviculales | Bacillariophyta | Chromobiota |
| Nannochloropsis | Monodopsidaceae | Eustigmatales | Heterokontophyta | Chromobiota |
| Pavlova | Pavlovaceae | Pavlovales | Haptophyta | Chromobiota |
| Isochrysis | Isochrysidaceae | Isochrysidales | Haptophyta | Chromobiota |

Phylogeny according to: http://www.algaebase.org/browse/taxonomy/
Note:
Many genes that in higher plants and Chlorophyta are encoded in the nucleus are encoded on the chloroplast genome (plastome) in the Chromobiota, red lineage algae (Grzebyk, et al., 2003)

This is done for the following cyanobacteria:_{—}Synechococcus PCC7002, Synechococcus WH-7803, Thermosynechococcus elongatus BP-1 as representatives of all cyanobacterial species

It is however, clear for one skilled in the art that this list is not exclusive, but that various other genera and species can be used as well.

The method is intended to confer resistance to viruses of the following groups among others: Viruses infecting green algae: Phycodnaviridae PBCV-1, Chlorovirus NC64A, Pbi, Chlorella virus CVK1, CVK2, NY-2A. Viruses (phage) infecting cyanobacteria: Podoviridae Cyanophages phil2. Myoviridae Cyanophages AS-1, Ma, Ma-HPM05, Ma-LMMO2, Ma-LMM03,S-BM4, S-BnM1, S-KM1, S-RIM, S-RSM28, S-RSM88, Synechococcus cyanophages syn 1, 9, 10, 19, 26, 30, 33, S-CBP1, S-CBM2, S-CBP3, S-CBP42, S-CBM17, S-CBM32, S-CBM66, S-CBM68, S-CBM8, S-PWM3, S-RSM2, S-WHM1. Cyanomyoviruses: PP, P1, P3, P5, P6, P8, P12, P16, P17, P39, P60, P61, P66, P73, P76, P77, P79, P81, Φ9, Φ12, S-PM2, P-SSM2, P-SSM4, S-BnM1, S-WHM1, S-PWM1, SBP1, SssRNAV as based on NCBI terminology. It is however, clear for one skilled in the art that this list is not exclusive, but that various other viruses/phages can be protected against, as well, including various single or double RNA and DNA stranded viruses.

In one embodiment, a novel method of choosing the genetic material conferring the resistance is described. It consists of viral DNA enzymatic restriction into fragments varying in size, cloning those fragments into appropriate algae/cyanobacteria vectors, transforming cultures with mixed fragments and selecting for resistant individuals by the viral overlay technique. This invention provides protection to the specific virus, using its genome fragments that are expressed in the algae/cyanobacteria, as well as cross protection to other virus species. This method is especially useful with poorly studied viruses/phage where there is little genomic annotation that would allow choosing fragments likely to confer resistance, or even any information on virus sequences. Moreover, this method allows identification of components/genes that confer virus resistance and were previously unknown.

### Examples

### General Description

To achieve resistance to different viruses the following steps are performed. Cloning of viral/phage fragments into the right algae/cyanobacteria expression vectors is conducted either from a cosmid library (for algae phage genome) or from pBluescript II KS+/Puc18 vector (for the cyanophage). Each construct is transformed into the appropriate algae/cyanobacteria. Selection of the transformed algae/cyanobacteria, harboring virus resistant, is made according to the method described in (Van Etten et al., 1983a) for algae and in (Wilson et al., 1993) for cyanobacteria.

The following examples refer to the Chlorella virus (PBCV-1) and the Synechococcus cyanophage (P60/Syn9), however these examples can be reproduced for other viruses/phages as well. The isolation of the virus DNA, its digestion and the transformations to algae/cyanobacteria is modified for each organism according to its needs, based on modifications of standard protocols.

### Example 1

### Sub-Cloning of PBCV-1 Virus Genomic DNAs into an Algae Expression Vector

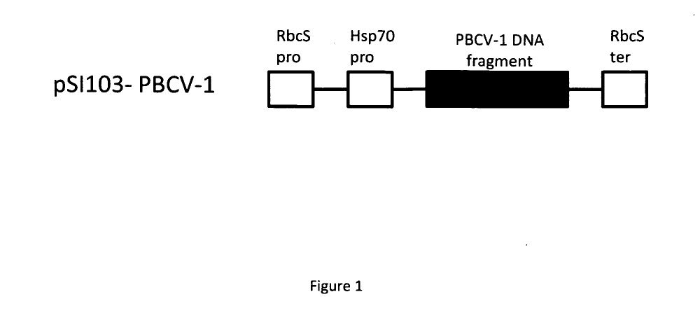

The growth of the PBCV-1 host, Chlorella on MBBM medium, the production and purification of PBCV-1 virus and the isolation of PBCV-1 DNA were described previously (Van Etten et al., 1981; Van Etten et al., 1983). A PBCV-1 DNA cosmid library was prepared and the cosmid insert DNAs were mapped to the PBCV genome as described (Li et al., 1995; Lu et al., 1995). The cosmid insert PBCV-1 DNAs are cloned under the control of the RbcS2-Hsp70 promoters and upstream to the 3′rbcS2 terminator, in the plasmid pSI103 (Sizova et. al 2001)(FIG. 1), as well as into various expression vectors, allowing various levels of expressions driven by different promoters.

### Example 2

### Transformation of the Virus Genomic Clones into Algae

Constructs are transformed using various techniques as described below

### I. Electroporation

Fresh algal culture are grown to mid exponential phase (2-5*10⁶ cells/ml) in artificial sea water (ASW)+F/2 media. Cells are then harvested and washed twice with fresh media. After resuspending the cells in 1/50 of the original volume, protoplasts are prepared by adding an equal volume of 4% hemicellulase (Sigma) and 2%, Driselase (Sigma), in ASW and incubating at 37° C. for 4 hours. Protoplast formation was tested as a lack of Calcofluor white (Fluka) staining of cell walls. Protoplasts are washed twice and with ASW containing 0.6M D-mannitol and 0.6M D-sorbitol and resuspended in the same media, after which DNA is added (10 μg linear DNA for each 100 μl protoplasts). Protoplasts are transferred to cold electroporation cuvettes and incubated on ice for 7 minutes then pulsed by the ECM 830 electroporator (BTX Instrument Division Harvard Apparatus, Inc. Holliston, Mass., USA). A variety of pulses are usually applied, ranging from 1000 to 1500 volts, 10-20 ms each pulse. Each cuvette was pulsed 5-10 times. Immediately after pulsing the cuvettes are placed on ice for 5 minutes and then the protoplasts are added to 250 μl of fresh growth media (without selection). After incubating the protoplasts for 24 hours in low light, the cells are plated onto selective solid media and incubated under normal growth conditions until single colonies appeared.

### II. Microporation

Fresh algal cultures are grown to mid exponential phase (2-5*10⁶ cells/ml) in ASW+F/2 media. A 10 ml sample of each culture was harvested, washed twice with DPBS (Dulbecco's phosphate buffered saline, Gibco) and resuspended in 250 μl of buffer R (supplied by Digital Bio, NanoEnTek Inc., Seoul, Korea, the producer of the microporation apparatus and kit). After adding 8 μg linear DNA to every 100 μl cells the cells are pulsed. A variety of pulses was usually needed, depending on the type of cells, ranging from 700 to 1700 volts, 10-40 ms pulse length; each sample was pulsed 1-5 times. Immediately after pulsing the cells are transferred to 200 μl fresh growth media (without selection). After incubating for 24 hours in low light, the cells are plated onto selective solid media and incubated under normal growth conditions until single colonies appeared.

### III. Particle Bombardment

Fresh algal culture are grown to mid exponential phase (2-5*10⁶ cells/ml) in ASW+F/2 media. 24 hours prior to bombardment cells are harvested, washed twice with fresh ASW+F/2 and resuspended in 1/10 of the original cell volume in ASW+F/2. 0.5 ml of the cell suspension is spotted onto the center of a 55 mm Petri dish containing solidified ASW+F/2 media. Plates are left to dry under normal growth conditions. Bombardment is carried out using a PDS1000/He biolistic transformation system according to the manufacturer's (BioRad Laboratories Inc., Hercules, Calif., USA) instructions using M10 tungsten powder (BioRad Laboratories Inc.) for cells larger than 2 microns in diameter, and tungsten powder comprised of particles smaller than 0.6 microns (FW06, Canada Fujian Jinxin Powder Metallurgy Co., Markham, ON, Canada) for smaller cells. The tungsten is coated with linear DNA. 1100 or 1350 psi rupture discs are used. All disposables are supplied by BioRad Laboratories Inc., (Hercules, Calif., USA). After bombardment the plates are incubated under normal growth conditions for 24 hours after which the cells are onto plated onto selective solid media and incubated under normal growth conditions until single colonies appear.

### IV. Glass Beads

Cells (4×10⁷) in 0.4 ml of growth medium containing 5% PEG6000 are transformed with DNA (1±5 mg) by the glass bead vortexing method (Kindle, 1990). The transformation mixture is then transferred to 10 ml of non-selective growth medium for recovery. The cells are kept for at least 18 h at 25° C. in the light. Cells are collected by centrifugation and plated at a density of 13×10⁷ cells per 80 mm plate. Transformants are selected on fresh SGII ((http://www.chlamy.org/SG.html). Agar plates containing the appropriate selection.

These procedures are carried out on the following algae: Chlamydomonas reinhardtii, Pavlova lutheri, Isochrysis CS-177, Nannochloropsis oculata CS-179, Nannochloropsis like CS-246, Nannochloropsis salina, Tetraselmis suecica, Tetraselmis chuii, and Nannochloris sp. as representatives of all algae species.

### Example 3

### Selection of the Transformed Virus Resistant Algae

Selection is made according to (Van Etten et al., 1983a). Briefly, transformants of chlorella are grown to a density of 2×10⁷ to 3×10⁷ algae per milliliter, concentrated by centrifugation, and resuspended in MBBIM (Van Etten et al., 1983a) at 38×10⁷ algae per milliliter. Two hundred microliters of algae (7.6×10⁷ algae) plus 100 μl of appropriate dilutions of PBCV-1 are added to 2.5 ml of 0.7 percent agar in MBBM (48° C. to 50° C.) and immediately overlaid on Petri plates containing 15 ml of MBBM plus 1.5 percent agar. The plates are then incubated at 25° C. in continuous light. Plaques are visible after 2-4 days. There is a linear relation between viral concentration, as measured by light scattering at A260, and number of plaques. Typically, 1.5 to 3×10′° plaque-forming units of PBCV-1 are obtained per unit of absorption at 260 nm (Van Etten et al., 1983a).

The resistant types of algae are isolated and newly cultured to examine their growth, compared to the wild type. In order to select 5 virus resistant strains containing different pieces of viral DNA, the resistant types of algae are sequenced to find the non redundant sequences. The 5 different strains are mixed in the feeder bioreactor and afterwards seeded to ponds where they are monitored to check that all five strains remain in the ponds on continuous dilution over time. The construction of the different virus genome fragments into the algal expression vectors, the transformation and selection for resistance protocols are modified for each organism according to its needs, based on modifications of standard protocols.

### Example 4

### Sub-cloning of Syn9/P60 Phage Genomic DNAs into a Cyanobacteria Expression Vector

The growth of the Syn9 host, Synechococcus strain WH8109 on SN medium, the production and purification of Syn9; and the isolation of Syn9 DNA were described previously (Weigele et al., 2007) The growth of the p60 host, Synechococcus strain WH7803, the production and purification of P60; and the isolation of P60 DNA were previously described (Lu et al., 2001; Chen and Lu, 2002).

Syn9 genomic library was constructed in the EcoRV site of the pBluescript II KS+vector, harboring fragments of blunt-ended DNA ranging from 1 to 3 kb, as described in (Weigele et al., 2007). P60 genomic library was constructed in the BamHI site of the pUC18 plasmid, as described in (Chen and Lu, 2002).

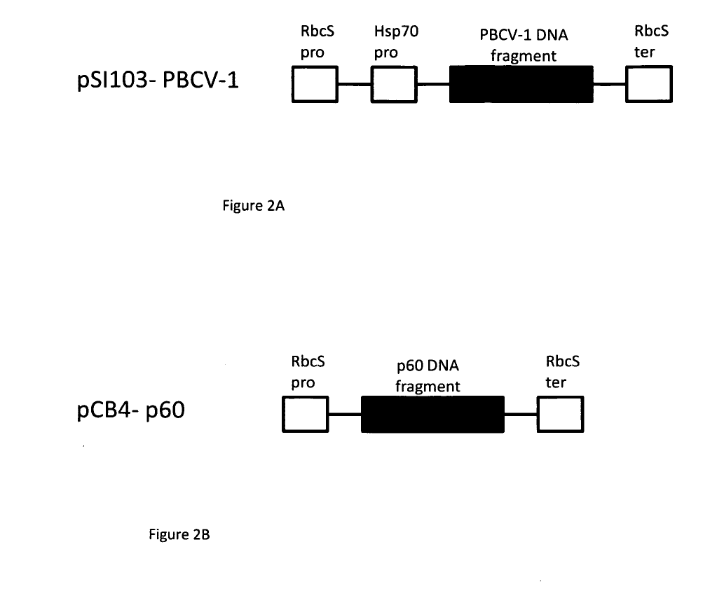

The Syn9 and p60 DNA fragments are cloned under the constitutive promoter of the rbcLS operon (Deng and Coleman, 1999) in the plasmid pCB4 (FIGS. 2A, 2B), as well as into various expression vectors, allowing various levels of expressions.

### Example 5

### Transformation of Phage Genomic Clones into Cyanobacteria

Constructs are incorporated into the cyanobacteria Synechococcus as set out in Golden et al., 1987. Briefly, cells are harvested by centrifugation and re-suspended in BG-11 medium at a concentration of 2-5×10⁸ cells per ml. To one ml of this cell solution the appropriate plasmid construct is added to a final concentration of 2-5 μg/ml. Cells are incubated in the dark for 8 hours followed by a 16 h light incubation prior to plating on BG-11 plates containing antibiotic. Plates were incubated under the standard growth conditions (30° C., light intensity of 100 μmol photons m⁻² s⁻¹). Antibiotic resistant colonies are visible in 7-10 days. This is modified for each organism according to its needs, based on modifications of standard protocols. In some cases antibiotic marker genes are omitted, and colonies are selected directly, without antibiotic preselection, as outlined in the following example. This is done to Synechococcus PCC7002, Synechococcus WH-7803, Thermosynechococcus elongatus BP-1 as representatives of all cyanobacterial species.

### Example 6

### Selection of the Transformed Phage Resistant Cyanobacteria

Plaque selection assay is performed as described in (Wilson et al., 1993) with pre-selection for a selectable marker other than phage (Example 9), or without such pre-selection. Serial dilutions of the cyanophage filtrates are added to separate 0.5-ml volumes of a 40× concentration (ca. 8×10⁹ cells ml⁻¹) of exponentially growing Synechococcus PCC7002 which are incubated at 25° C. for 1 h with occasional agitation to encourage cyanophage adsorption. Each phage-cell suspension is then added to 2.5 ml of 0.4% molten ASW agar (42° C.); these suspensions are mixed gently and then poured evenly onto a solid 1% ASW agar plate (diameter, 85 mm) before being left to set at room temperature for 1 h. Incubation of the plates is carried out at 25° C. under constant illumination (15 to 25 μmol m⁻² s⁻¹), and the plates are monitored daily for the formation of plaques. Control plates receive no cyanophages addition.

In order to select 5 strains containing different pieces of viral DNA that are virus resistant, the resistant types of cyanobacteria are sequenced to find the non redundant sequences. The 5 different strains are mixed in the feeder bioreactor and afterwards send to ponds where they are monitored to check that all five strains remain in the ponds on continuous dilution over time. The construction of the different phage genomes into the cyanobacterial expression vectors, the transformation and selections for resistance protocols are modified for each organism according to its needs, based on modifications of standard protocols.

### Example 7

### Sub-Cloning, Transformation and Selection of Algae that Confer Schizochytrium RNA Virus Resistance

The growth of the Schizochytrium single-stranded RNA virus (SssRNAV), the production and purification of SssRNAV RNA are as described previously (Yoshitake et. al., 2006). The viral RNA is used to synthesize cDNA as a template. First strand synthesis is performed by using the SuperScript reverse transcriptase for cDNA synthesis (Invitrogen) according to the manufacturer's instructions, using both oligo (dT)12-18 primers and random hexamers. A second strand cDNA synthesis is performed using DNA polymerase I and RNase H (Fermentas), according to the manufacturer's instructions. Purified dsDNA products are used for further cloning into pGEM-T easy vector (Promega). The various reverse transcribed viral cDNA fragments from pGEM-T easy are cloned under the control of the RbcS2-Hsp70 promoters and upstream to the 3′rbcS2 terminator, in the plasmid pSI103 (Sizova et. al 2001), as well as into various expression vectors, allowing various levels of expressions driven by different promoters. Transformation is conducted according to example 2 and selection of algae harboring the RNA virus resistance is preformed as detailed in example 3. The construction of the different RNA virus genomes into the algae expression vectors, the transformation and selections for resistance protocols are modified for each organism according to its needs, based on modifications of standard protocols.

### REFERENCES

Ackermann H W, DuBow M S (1987). Viruses of Prokaryotes, Vol I and II. CRC Press, Boca Raton, Fla.

Bailey S, Clokie M R J, Millard A, Mann N H (2004) Cyanophage infection and photoinhibition in marine cyanobacteria. Research in Microbiology 155: 720-725

Baulcombe D C (1996) Mechanisms of pathogen-derived resistance to viruses in transgenic plants. Plant Cell 8: 1833-1844

Bratbak G, Egge, J. K. Heldal, M. (1993) Viral mortality of the marine alga Emiliana huxleyi (Haptophyceae) and termination of algal blooms. Appl. Environ. Microbiol. 66: 4916-4920

Chen F, Lu J (2002) Genomic sequence and evolution of marine cyanophage P60: a new insight on lytic and lysogenic phages. Appl. Environ. Microbiol. 68: 2589-2594

Cochran P K, Paul J H (1998) Seasonal Abundance of Lysogenic Bacteria in a Subtropical Estuary. Appl. Environ. Microbiol. 64: 2308-2312

Deng M D, Coleman J R (1999) Ethanol synthesis by genetic engineering in cyanobacteria. Appl Environ Microbiol 65: 523-528

Golden S S, Brusslan J, Haselkorn R (1987) Genetic engineering of the cyanobacterial chromosome. Methods Enzymol 153: 215-231

Grzebyk, D., O. Schofield, P. Falkowski, and J. Bernhard (2003) The Mesozoic radiation of eukaryotic algae: the portable plastid hypothesis. J. Phycol. 39:259-267)

Jacobsen A G, Bratbak Heldal M (1996). Isolation and characterization of a virus infecting Phaeocystis pouchetii (Prymnesiophyceae). J. Phycol. 32:923-927

Jiang S C, Paul J H (1998) Significance of lysogeny in the marine environment: studies with isolates and a model of lysogenic phage production. Microbial Ecology 35: 235-243

Jiang S C P, J. H. (1996) Occurrence by lysogenic bacteria in marine microbial communities as determined by prophage induction. Mar. Ecol. Prog. Ser. 142: 27-38

Kindle, K, L., (1990). High-frequency nuclear transformation of Chlamydomonas reinhardtii. Proc. Natl. Acad. Sci. USA 87: 1228-1232.

Li Y, Lu Z, Burbank D E, Kutish G F, Rock D L, Van Etten J L (1995) Analysis of 43 kb of the Chlorella Virus PBCV-1 330-kb Genome: Map Positions 45 to 88. Virology 212: 134-150

Lindbo J A, Dougherty W G (1992) Untranslatable transcripts of the tobacco etch virus coat protein gene sequence can interfere with tobacco etch virus replication in transgenic plants and protoplasts. Virology 189: 725-733

Lu J, Chen F, Hodson R E (2001) Distribution, isolation, host specificity, and diversity of cyanophages infecting marine Synechococcus spp. in river estuaries. Applied and Environmental Microbiology 67: 3285-3290

Lu Z, Li Y, Zhang Y, Kutish G F, Rock D L, Van Etten J L (1995) Analysis of 45 kb of DNA located at the left end of the Chlorella virus PBCV-1 genome. Virology 206: 339-352

Marston M F, Sallee J L (2003) Genetic diversity and temporal variation in the cyanophage community infecting marine Synechococcus species in Rhode Island's coastal waters. Appl. Environ. Microbiol. 69: 4639-4647

McDaniel L, Houchin L A, Williamson S J, Paul J H (2002) Plankton blooms: Lysogeny in marine Synechococcus. Nature 415: 496-496

Milligan K L D, Cosper E M (1994) Isolation of virus capable of lysing the brown tide microalga, Aureococcus anophagefferens. Science 266: 805-807

Short C M, Suttle C A (2005) Nearly identical bacteriophage structural gene sequences are widely distributed in both marine and freshwater environments. Appl. Environ. Microbiol. 71: 480-486

Sizova, I., et al. (2001). A Streptomyces rimosus aphVIII gene coding for a new type phosphotransferase provides stable antibiotic resistance to Chlamydomonas reinhardtii. Gene 277: 221-229.

Stam M, Mol J N M, Kooter J (1997) The silence of genes in transgenic plants. Annals of Botany 79: 3

Tapper M A H, R. E. (1998) Temperate viruses and lysogeny in Lake Superior bacterioplankton. Limnol. Oceanogr. 43: 95-103

Tung K-C C W L (1999) Electrotransformation of Chlorella vulgaris. Plant Cell Reports 18: 778-780

Van Etten J L, Meints R H, Burbank D E, Kuczmarski D, Cuppels D A, Lane L C (1981) Isolation and characterization of a virus from the intracellular green alga symbiotic with Hydra viridis. Virology 113: 704-711

Van Etten J L, Burbank D E, Kuczmarski D, Meints R H (1983a) Virus infection of culturable Chlorella-like algae and development of a plaque assay. Science 219: 994-996

Van Etten J L, Burbank D E, Xia Y, Meints R H (1983b) Growth cycle of a virus, PBCV-1, that infects Chlorella-like algae. Virology 126: 117-125

Waterbury J B, Valois F W (1993) Resistance to co-occurring phages enables marine Synechococcus communities to coexist with cyanophages abundant in seawater. Appl. Environ. Microbiol. 59: 3393-3399

Weigele P R, Pope W H, Pedulla M L, Houtz J M, Smith A L, Conway J F, King J, Hatfull G F, Lawrence J G, Hendrix R W (2007) Genomic and structural analysis of Syn9, a cyanophage infecting marine Prochlorococcus and Synechococcus. Environmental Microbiology 9: 1675-1695

Wilson W H, Joint I R, Carr N G, Mann N H (1993) Isolation and Molecular Characterization of five marine cyanophages propagated on Synechococcus sp. strain WH7803. Applied and Environmental Microbiology 59: 3736

Wommack K E, Colwell R R (2000) Virioplankton: Vairuses in aquatic ecosystems. Microbiol. Mol. Biol. Rev. 64: 69-114

Yoshitake T, Kazuyuki M, Keizo N, Tetsuro O, Daiske H (2006) Complete nucleotide sequence and genome organization of a single-stranded RNA virus infecting the marine fungoid protist Schizochytrium sp. Journal of General Virology 87: 723-733.

Zhong Y, Chen F, Wilhelm S W, Poorvin L, Hodson R E (2002) Phylogenetic diversity of marine cyanophage isolates and natural virus communities as revealed by sequences of viral capsid assembly protein gene g20. Appl. Environ. Microbiol. 68: 1576-1584

What is claimed is:

1. A method to render algae or cyanobacteria resistant to infections caused by a previously known or unknown algae and cyanobateria virus and bacteriophage, said method comprising the steps of:
a) restricting algae and cyanobateria viral or phage DNA or cDNA into fragments by DNA enzymatic restriction;
b) bulk cloning all the fragments into algal or cyanobacterial vectors;
c) transforming the algal or cyanobacterial cultures with the vectors; and
d) selecting resistant individuals by algae or cyanobacteria virus or phage resistance screen.

2. The method of claim 1, wherein the virus is an RNA virus, and the method includes a step of reverse transcribing the virus RNA to cDNA.

3. The method of claim 1, wherein the transformation is performed through microporation.

4. The method of claim 2, wherein the transformation is performed through microporation.

5. The method of claim 1, wherein the alga is selected from the group consisting of Chlamydomonas reinhardtii, Chlorella sp. Pavlova lutheri, Isochrysis CS-177, Nannochloropsis CS-179, Nannochloropsis CS-246, Nannochloropsis salina CS-190, Tetraselmis suecica, Tetraselmis chuii and Nannochloris sp.

6. The method of claim 5, wherein PBCV-1 virus DNA is restricted into fragments and cloned in the plasmid pSI103 under RbcS2-Hsp70 promoters.

7. The method of claim 2, wherein SssRNAV RNA virus is reverse transcribed into cDNA, followed by double stranded DNA synthesis and cloned in the plasmid pSI103 under RbcS2-Hsp70 promoters.

8. The method of claim 1, wherein the cyanobacterium is selected from the group consisting of Synechococcus PCC7002, Synechococcus WH-7803, and Thermosynechococcus elongatus BP-1.

9. The method of claim 8, wherein Syn9 DNA is restricted into fragments and cloned in pCB4 plasmid under rbcLS promoters.

10. The method of claim 8, wherein P60 is restricted into fragments and cloned in pCB4 plasmid under rbcLS promoters.

11. The method of claim 1, wherein there is no previous information about which virus/phage sequences are likely to confer resistance.

12. A method to prevent virus or bacteriophage contamination of algal or cyanobacterial culture in bioreactor, said method comprising the steps of:
a. Rendering algae or cyanobacteria resistant according to the method of claim 1; and
b. Cultivating several resistant strains of one algal/cyanobacterial species together in bioreactor, whereby the culture is taken over by a resistant strain when a viral/phage pathogen infects it.

13. A algae or cyanobacteria culture conferring enhanced resistance against single or double stranded DNA or RNA algae and cyanobacteria virus or bacteriophage infections, said culture comprising various strains of algae or cyanobacteria that are made resistant against various pathogens through the method of claim 1.

14. A method to provide protection to a specific virus or bacteriophage and cross protection to other viruses and bacteriophages, said method comprising cultivation of various strains of resistant algae or cyanobacteria of claim 1.

15. A method to identify new components or genes conferring virus resistance, said method comprising the steps of :
a) restricting viral or phage DNA or cDNA into fragments by DNA enzymatic restriction;
b) cloning the mixed fragments into algal or cyanobacterial vectors;
c) transforming algae or cyanobacteria cultures with fragments;
d) selecting resistant individuals by virus resistance screen; and
e) characterizing the fragments inducing the resistance.
